(19) United States
(12) Patent Application Publication
(10) Pub. No.: US 2005/0085616 A1
(43) Pub. Date: Apr. 21, 2005

(54) Aqueous dispersions consisting of polycarbodimides

(75) Inventors: Ulrike Licht, Mannheim (DE); Karl Haberle, Speyer (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/502,650

(22) PCT Filed: Feb. 4, 2003
(86) PCT No.: PCT/EP03/01055

(51) Int. Cl. C08G 18/10; C08G 18/32; C07C 267/00

(52) U.S. Cl. 528/61; 564/252

(57) ABSTRACT

Aqueous dispersions composed of polycarbodiimides which carry substantially no carboxyl groups and I are obtained from polyisocyanates of the formula I
OCN—(R^{c}—N═C═N)ₙ—R^{c}—NCO (I)
where R^{c} is a divalent hydrocarbon radical with or without urea, urethane, ester and/or ether groups, as is obtained by removal of the isocyanate groups from a simple organic isocyanate or from a prepolymer which contains urethane groups and, if desired, ether or ester groups and which carries terminal isocyanate groups, two or more radicals R^{c} present in the same molecule having identical or different definitions, and n being from 1 to 20, or II are of the formula (II)
   R^{d}—(R^{c}—N═C═N—R^{c})ₙ—R^{d} (II) where n is a number from 1 to 20,
R^{c} is as defined for the formula (I),
R^{d} is —NH₂ 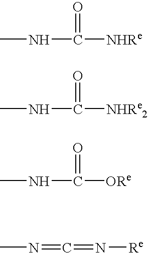
and R^{e} independently at each occurrence is C₁ to C₂₀ alkyl or alkenyl, C₅ to C₁₂ cycloalkyl, C₆ to C₂₀ aryl or C₇ to C₂₀ aralkyl, in which 1 to 4 hydrogen atoms of the radical R^{e} may be substituted by substituents that are inert toward carbodiimide groups and in which the radical R^{e} ought not to contain any hydrophilic groups capable of producing dispersibility in water.

The present invention relates to aqueous dispersions composed of polycarbodiimides which carry substantially no carboxyl groups and

I are obtained from polyisocyanates of the formula I
OCN—(R^{c}—N═C═N)ₙ—R^{c}—NCO (I)
where R^{c} is a divalent hydrocarbon radical with or without urea, urethane, ester and/or ether groups, as is obtained by removal of the isocyanate groups from a simple organic isocyanate or from a prepolymer which contains urethane groups and, if desired, ether or ester groups and which carries terminal isocyanate groups, two or more radicals R^{c} present in the same molecule having identical or different definitions, and n being from 1 to 20, or

I are of the formula (II)
R^{d}—(R^{c}—N═C═N—R^{c})ₙ—R^{d} (II)
where
n is a number from 1 to 20,
R^{c} is as defined for the formula (I), 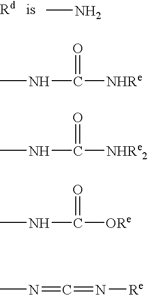
and R^{e} independently at each occurrence is C₁ to C₂₀ alkyl or alkenyl, C₅ to C₁₂ cycloalkyl, C₆ to C₂₀ aryl or C₇ to C₂₀ aralkyl, in which 1 to 4 hydrogen atoms of the radical R^{e} may be substituted by substituents that are inert toward carbodiimide groups and in which the radical R^{e} ought not to contain any hydrophilic groups capable of producing dispersibility in water.

The present invention further relates to a process for preparing the aqueous dispersions of the invention, to their use as a constituent of binders in adhesives, varnishes, paints, papercoating slips or fiber nonwovens, and to articles of wood, metal, textile, leather or plastic that have been treated with the aqueous dispersion of the invention.

Carbodiimide groups react with carboxylic acids to give an N-acylurea compound and are therefore highly suitable as crosslinkers for polymers containing carboxylic acid groups. For this reason, one of the uses of polycarbodiimides is as crosslinkers of carboxylate-containing latices (U.S. Pat. No. 4,419,294; U.S. Pat. No. 4,820,863).

U.S. Pat. No. 4,977,219 and U.S. Pat. No. 5,117,059 disclose mixtures of an aqueous dispersion of a carbodiimide and an aqueous dispersion of an emulsion polymer containing carboxylate groups, the former dispersion being stabilized with the aid of customary surface-active substances.

U.S. Pat. No. 5,574,083 relates to a mixture of an aqueous dispersion of carbodiimides, the dispersion being stabilized by hydrophilic polyalkylene oxide radicals carried by the carbodiimides. These dispersions are blended with aqueous dispersions of an emulsion polymer containing carboxylate groups.

EP-A 686626 discloses adding water-soluble carbodiimide crosslinkers to the aqueous phase of emulsion polymers. A disadvantage of this procedure is that the carbodiimides must first be brought into a water-soluble form by reaction, for example, with ionic compounds. Moreover, hydrophilic polycarbodiimides have the disadvantage of inadequate stability on storage and the disadvantage that they give the films crosslinked using them a permanent hydrophilicity which is accompanied by an unwanted low water resistance.

Hydrophobic polycarbodiimides, on the other hand, are not obtainable as dispersions or emulsions having long-term stability, but instead must first be incorporated into the target dispersion using solvents, which means an additional complex step for the user.

It is an object of the present invention to remedy the disadvantages depicted above and to develop novel aqueous dispersions which comprise carbodiimides, can be kept even for months without loss of crosslinker activity, and can be processed into water-resistant crosslinked films.

We have found that this object was achieved by the aqueous dispersions defined at the outset and by a process for preparing them. The present invention further extends to the use of the aqueous dispersions as a constituent of binders in adhesives, varnishes, paints, papercoating slips or fiber nonwovens, and also to articles of wood, metal, textile, leather or plastic that have been treated with such aqueous dispersions.

The aqueous dispersions of the invention may be composed of polycarbodiimides which carry substantially no carboxyl groups and are obtained from polyisocyanates of the formula I
OCN—(R^{c}—N═C═N)ₙ—R^{c}—NCO (I)
where R^{c} is a divalent hydrocarbon radical with or without urea, urethane, ester and/or ether groups, as is obtained by removal of the isocyanate groups from a simple organic isocyanate or from a prepolymer which contains urethane groups and, if desired, ether or ester groups and which carries terminal isocyanate groups, two or more radicals R^{c} present in the same molecule having identical or different definitions, and n being from 1 to 20.

The polycarbodiimides are preferably obtained from polyisocyanates of the formula I where n is a number from 2 to 10 and R^{c} is a divalent hydrocarbon radical containing from 2 to 50 carbon atoms. With particular preference R^{c} is a divalent hydrocarbon radical containing from 4 to 20 carbon atoms and without further functional groups, such as urea, urethane, ester or ether groups, for example. Very particular preference is given to polycarbodiimides derived from polyisocyanates of the formula I where R^{c} is a group of the formula (I′) 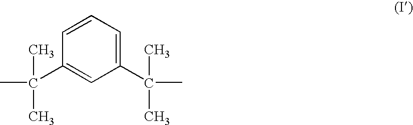
(tetramethylxylylene group).

In the formula I the radicals R^{c} are preferably derived by abstracting the isocyanate groups from monomers which are diisocyanates commonly used in polyurethane chemistry.

Generally speaking, carbodiimide groups are readily obtainable from two isocyanate groups with elimination of carbon dioxide:

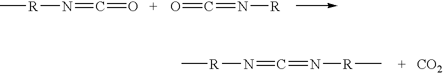

Starting from diisocyanates, it is possible in this way to obtain oligomeric compounds containing two or more carbodiimide groups and, where appropriate, isocyanate groups, especially terminal isocyanate groups.

The isocyanate groups still present may be reacted further, for example, with alcohols, thiols or primary or secondary amines to form urethane, thiourethane or urea groups. The alcohols, thiols and primary and secondary amines preferably contain no functional groups other than hydroxyl groups, thiol groups and primary and secondary amino groups. The carbodiimides may therefore contain isocyanate groups and the above reaction products thereof.

Preference is given to polycarbodiimides obtainable from polyisocyanates, especially diisocyanates, with elimination of carbon dioxide.

The polycarbodiimides for use in accordance with the invention are preferably prepared by condensing polyisocyanates of the formula I at temperatures from 50 to 200° C., in particular from 100 to 190° C., with evolution of carbon dioxide.

Suitable diisocyanates include preferably diisocyanates X(NCO)₂ where X is an aliphatic hydrocarbon radical having from 4 to 12 carbon atoms, a cycloaliphatic or aromatic hydrocarbon radical having from 6 to 15 carbon atoms or an araliphatic hydrocarbon radical having from 7 to 15 carbon atoms. Examples of such diisocyanates are tetramethylene diisocyanate, hexamethylene diisocyanate, dodecamethylene diisocyanate, 1,4-diisocyanatocyclohexane, 1-isocyanato-3,5,5-trimethyl-5-isocyanatomethylcyclohexane (IPDI), 2,2-bis(4-isocyanatocyclo-hexyl)propane, trimethylhexane diisocyanate, 1,4-diisocyanato-benzene, 2,4-diisocyanatotoluene, 2,6-diisocyanatotoluene, 4,4′-diisocyanatodiphenylmethane, 2,4′-diisocyanatodiphenyl-methane, p-xylylene diisocyanate, tetramethylxylylene diisocyanate (TMXDI), the isomers of bis(4-isocyanatocyclo-hexyl)methane (HMDI) such as the trans/trans, cis/cis, and cis/trans isomer, and mixtures of these compounds.

The preparation of the polycarbodiimides from diisocyanates is conventional and is described, for example, in U.S. Pat. No. 2,840,589, U.S. Pat. No. 2,941,966, and EP-A 628541. Polycarbodiimides of the formula I may also be prepared particularly gently absent of byproducts by a heterogeneous catalysis in accordance with DE-A 2 504 400 and DE-A 2 552 350. The carbodiimidization of diisocyanates in the presence of very small amounts of phospholene oxide with subsequent blocking of the catalyst using acid chlorides is subject matter, inter alia, of DE-A 2 653 120.

The aqueous dispersions of the invention may also be composed of polycarbodiimides which carry substantially no carboxyl groups and are of the formula (II)
R^{d}—(R^{c}—N═C═N—R^{c})ₙ—R^{d} (II)
where
n is a number from 1 to 20,
R^{c} is as defined for the formula (I), 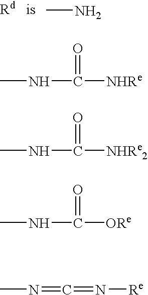
and R^{e} independently at each occurrence is C₁ to C₂₀ alkyl or alkenyl, C₅ to C₁₂ cycloalkyl, C₆ to C₂₀ aryl or C₇ to C₂₀ aralkyl, in which 1 to 4 hydrogen atoms of the radical R^{e} may be substituted by substituents that are inert toward carbodiimide groups and in which the radical R^{e} ought not to contain any hydrophilic groups capable of producing dispersibility in water. “Hydrophilic groups” are groups capable of penetrating the aqueous phase and remaining therein.

The aqueous dispersions of the invention preferably comprise polycarbodiimides wherein R^{c} in the formula II is a divalent hydrocarbon radical containing from 2 to 50 carbon atoms, in particular from 4 to 20 carbon atoms. With very particular preference, R^{c} is a radical of the formula I′

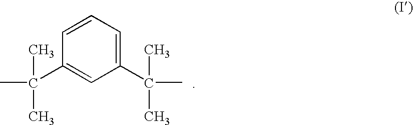

Polycarbodiimides used with preference contain a urethane bridge in particular as radical R^{d} and a C₁-C₂₀ alkyl or alkenyl bridge in particular as radical R^{e}.

Such polycarbodiimides of the formula II are normally prepared by eliminating carbon dioxide from two isocyanate groups, starting from suitable diisocyanates, in analogy to the preparation of the polycarbodiimides starting from polyisocyanates of the formula I.

In the aqueous dispersions it is advisable to use polycarbodiimides of the formula II which contain no ionic groups or no polyalkylene oxide groups having more than 5 ethylene oxide units.

The aqueous dispersions of the invention are preferably obtainable by a process which comprises first preparing a polycarbodiimide by heating a suitable diisocyanate at temperatures of from 50 to 200° C., in particular from 100 to 190° C., with elimination of carbon dioxide and then heating said polycarbodiimide at temperatures from 20 to 100° C., in particular from 60 to 80° C., to give a highly mobile melt. The resulting melt is subsequently emulsified in water with the aid of suitable emulsifiers or protective colloids. It is also possible to use water-immiscible solvents which following emulsification are distilled off or remain in the product. These solvents are preferably not used.

Emulsification in water produces emulsified monomer droplets having a diameter of up to 50 μm; the carbodiimides are in solution and/or dispersion in these monomer droplets.

One of the features of the aqueous dispersions of the invention is that the polycarbodiimides can be present in the form of a miniemulsion in water, which means that the polycarbodiimides are present in water in emulsified particles with a diameter of up to 5 μm, in particular of up to 1 μm. To prepare the miniemulsion, the particles in which the polycarbodiimides are located are subjected to severe shearing, and are dispersed as a result. Severe shearing of this kind may be achieved by methods including high-pressure homogenization, ultrasound, or jet dispersers. It is preferred here to operate using ultrasound.

From the melt obtained by heating the polycarbodiimide it is preferred to prepare a preemulsion in water containing an effective amount of emulsifiers and/or protective colloids. The emulsion is then prepared, normally using ultrasound or a high-pressure homogenizer. This is done preferably at temperatures from 20 to 100° C., in particular from 60 to 80° C., and is guided by the viscosity of the melt and the sensitivity of the emulsion.

The polycarbodiimides are normally emulsified in water with the aid of suitable emulsifiers and/or protective colloids. As is normal in the case of emulsion polymerization, the surface-active substances used include ionic and/or nonionic emulsifiers and/or protective colloids or stabilizers.

A detailed description of suitable protective colloids is given in Houben-Weyl, Methoden der organischen Chemie, volume XIV/1, Makromolekulare Stoffe, Georg-Thieme-Verlag, Stuttgart, 1961, pp. 411 to 420. Anionic, cationic, and nonionic emulsifiers are suitable. As accompanying surface-active substances it is preferred to use exclusively emulsifiers, whose molecular weights, unlike those of the protective colloids, are normally below 2000 g/mol. Where mixtures of surface-active substances are used the individual components must of course be compatible with one another, which in case of doubt can be checked by means of a few preliminary tests. As surface-active substances it is preferred to use anionic and nonionic emulsifiers. Examples of customary accompanying emulsifiers are ethoxylated fatty alcohols (EO units: 3 to 50, alkyl; C₈ to C₃₆), ethoxylated mono-, di-, and trialkylphenols (EO units: 3 to 50, alkyl: C₄ to C₉), alkali metal salts of dialkyl esters of sulfosuccinic acid and also alkali metal salts and ammonium salts of alkyl sulfates (alkyl: C₈ to C₁₂), of ethoxylated alkanols (EO units: 4 to 30, alkyl: C₁₂ to C₁₈), of ethoxylated alkylphenols (EO units: 3 to 50, alkyl: C₄ to C₉), of alkylsulfonic acids (alkyl: C₁₂ to C₁₈) and of alkylarylsulfonic acids (alkyl: C₉ to C₁₈).

Suitable emulsifiers can also be found in Houben-Weyl, op. cit. pages 192 to 208.

Examples of trade names of suitable emulsifiers include Dowfax® 2 A1, Emulan® NP 50, Dextrol® OC 50, Emulgator 825, Emulgator 825 S, Emulan® OG, Texapon® NSO, Nekanil® 904 S, Lumiten® I-RA, LumitenE 3065 or Steinapol® NLS.

The surface-active substance is normally used in amounts from 0.1 to 10% by weight, based on the oil phase.

In the warming of the polycarbodiimide to a highly mobile melt it may be advisable to admix it with an additional hydrophobic, i.e., water-insoluble, material characterized by a water solubility of <10-⁵ g/l, preferably of <10-⁷ g/l. The hydrophobic material may be added in an amount from 0.01 to 10% by weight, in particular from 0.05 to 0.5% by weight. Examples of such hydrophobic materials include hydrocarbons such as hexadecane, halogenated hydrocarbons such as fluorinated hydrocarbons, silanes, organosilanes, siloxanes, long-chain esters, oils such as vegetable oil, hydrophobic dye molecules, blocked isocyanates, and oligomeric products of addition polymerization, polycondensation, and polyaddition.

In the case of the aqueous dispersion of the invention, suitable hydrophobic materials also include relatively high molecular mass representatives from the oligomer distribution of the substance to be emulsified, so that a foreign hydrophobic material is unnecessary. Likewise suitable as hydrophobic materials are structures of the formulae I or II in which the radicals R^{c} and/or R^{e} each stand for a long-chain alkane, particularly an alkane having from 10 to 20 carbon atoms.

It may further be advisable to react the polycarbodiimide prepared from the diisocyanate with an alcohol, to give a polyurethane containing carbodiimide groups. This can be done at temperatures from 20 to 120° C.,in particular from 60 to 80° C., and produces what is known as a polycarbodiimideurethane.

Alcohols suitable for this purpose are on the one hand low molecular mass monoalcohols or else diols having a molecular weight of about 32 to 500, in particular from 62 to 300 g/mol. It is preferred to use short-chain monoalcohols, i.e., branched or unbranched monoalcohols having from 6 to 30 carbon atoms, such as 1-hexanol, 2-ethylhexanol, or else dodecanol, behenol, and also stearyl acohol or oleyl alcohol.

Furthermore, the resulting polycarbodiimides may also be reacted with relatively high molecular mass alcohols having a molecular weight of from about 500 to 5000, preferably from about 1000 to 3000, g/mol. Suitable alcohols in this case include polyesterpolyols, polyetherdiols or else polyhydroxyolefins.

The reaction of the polycarbodiimide with the alcohols may be catalyzed using organometallic tin compounds, such as dibutyltin dilaurate, for example.

The aqueous dispersions of the invention react effectively with carboxylic acid groups and so are suitable as crosslinker systems for carboxylate-containing polymers. The aqueous dispersions can also be kept for months, i.e., are stable on storage, and can be processed with aqueous carboxyl-containing polymers to give water-resistant, crosslinked films. They are easy to prepare without great complexity.

The aqueous dispersions of the invention are particularly suitable as constituents of binders for coating compositions or impregnating compositions, e.g., for adhesives, varnishes, paints, papercoating slips or fiber nonwovens, i.e., in all cases where crosslinking and an increase in the internal strength (cohesion) are desired.

Depending on the intended use, the aqueous dispersion may comprise additives such as thickeners, flow assistants, pigments or fillers, fungicides, etc.

When used as adhesives, the aqueous dispersions of the invention may comprise not only the abovementioned additives but also specific auxiliaries and additives that are customary in adhesives technology. Examples of these include thickeners, plasticizers or else tackifying resins such as natural resins or modified resins such as rosin esters or synthetic resins such as phthalate resins, for example.

Polymer dispersions used as adhesives contain with particular preference alkyl (meth)acrylates as principal monomers of the polymer. Preferred applications in the adhesives field also include use as laminating adhesives, e.g., for lamination of composites and for high-gloss film lamination (adhesive bonding of transparent films to paper or card).

The aqueous dispersions of the invention may be applied by conventional methods to the substrates to be coated or impregnated.

The aqueous dispersions of the invention may be used in particular for adhesively bonding, impregnating or coating articles made of wood, metal, textile, leather or plastics.

### EXAMPLES

I. Preparation of a Diisocyanatocarbodiimide from TMXDI

750 parts by weight (3.1 mol) of 1,3-bis(1-methyl-1-isocyanato-ethyl)benzene (TMXDI) having an NCO content of 34.4% by weight were heated to 180° C. in the presence of 1.5 parts by weight, based on isocyanate, of 1-methyl-2-phospholene 1-oxide and condensed at this temperature with moderate evolution of carbon dioxide. After the reaction mixture had reached an NCO content of about 11% by weight, residues of unreacted TMXDI were distilled off under reduced pressure at a temperature of 180° C.

This gave a mixture of carbodiimides having an NCO content of 8.0% by weight, an —N═C═N group content of about 15% by weight, a melting point <30° C., and an iodine color number of from 5 to 7 as measured in accordance with DIN 6162.

II. Preparation of Polycarbodiimideurethanes

### Example 1

### Polycarbodiimideurethane with Oleyl Alcohol

In a stirring flask, 300 g of TMXDI-CDI, i.e., the polycarbodiimide from I, 180 g of oleyl alcohol and 0.1 g of dibutyltin dilaurate (DBTL) were stirred at 100° C. for 240 minutes. The NCO content of the viscous oil obtained was determined by titrimetry as being 0.03% by weight.

### Example 2

### Polycarbodiimideurethane with Stearyl Alcohol

In a stirring flask, 519 g of TMXDI-CDI, i.e., the polycarbodiimide from I, 257 g of stearyl alcohol and 0.1 g of DBTL were stirred at 100° C. for 240 minutes. The NCO content of the viscous oil obtained was determined by titrimetry as being 0.01% by weight.

### Example 3

### Polycarbodiimideurethane with Methyl Diglycol

In a stirring flask, 800 g of TMXDI-CDI, i.e., the polycarbodiimide from I, 182 g of methyl diglycol and 0.1 g of DBTL were stirred at 140° C. for 120 minutes. The NCO content of the viscous oil obtained was determined by titrimetry as being 0.01% by weight.

### Example 4

### Polycarbodiimideurethane with Ethylhexanol

In a stirring flask, 300 g of TMXDI-CDI, i.e., the polycarbodiimide from I, 74.4 g of 2-ethylhexanol and 0.1 g of DBTL were stirred at 100° C. for 180 minutes. The NCO content of the viscous oil obtained was determined by titrimetry as being 0.05% by weight.

III. Preparation of the Miniemulsion (Aqueous Dispersion)

Miniemulsion 1a

50 g of the polycarbodiimideurethane from example 1 were heated to 70° C. and mixed with 200 g of hot DI water containing 3 g of 15% Steinapol NLS (Na lauryl sulfate) as emulsifier. Using a magnetic stirrer rod, the mixture was stirred in a water bath at 70° C. for 5 minutes and then sonicated in a hot water bath using an ultrasonic rod (Branson Sonifier) for 10 minutes. After filtration through a 40 μm filter, the particle size was determined as being 300 nm.

40 Miniemulsion 1b

100 g of the polycarbodiimideurethane from example 1 were heated to 70° C., mixed with 2.5 g of oleyl alcohol and with 400 g of hot DI water containing 6 g of 15% Steinapol NLS as emulsifier. The mixture was homogenized in a water bath at 70° C. using an Ultraturrax (IKA T25, 24 000 rpm) for 3 minutes and cleaned by means of a 125 μm pressure filter. The preemulsion was then passed 3 times without cooling through a high-pressure homogenizer (from Microfluidics). After filtration through a 40 μm filter the particle size was determined as being 316 nm.

Miniemulsion 2

50 g of the polycarbodiimideurethane from example 2 were heated to 70° C. and mixed with 1.25 g of hexadecane and stirred at 70° C. for 1 hour. This was followed by the addition of 200 g of hot DI water containing 3 g of 15% Steinapol NLS as emulsifier. Using a magnetic stirrer rod, the mixture was stirred in a water bath at 70° C. for 5 minutes and then sonicated in a hot water bath using an ultrasonic rod (Branson Sonifier) for 10 minutes. After filtration through a 40 μm filter, the particle size was determined as being 321 nm.

Miniemulsion 3

50 g of the polycarbodiimideurethane from example 3 were heated to 70° C. and mixed with 2.5 g of the urethane from example 1; this was followed by the addition of 200 g of hot DI water containing 3 g of 15% Steinapol NLS as emulsifier. Using a magnetic stirrer rod, the mixture was stirred in a water bath at 70° C. for 5 minutes and then sonicated in a hot water bath using an ultrasonic rod (Branson Sonifier) for 10 minutes. After filtration through a 40 μm filter, the particle size was determined as being 350 nm.

Miniemulsion 4

50 g of the polycarbodiimideurethane from example 4 were heated to 70° C. and mixed with 1.25 g of hexadecane and stirred at 70° C. for 1 hour. This was then mixed with 200 g of DI water containing 3 g of 15% Steinapol NLS as emulsifier. Using a magnetic stirrer rod, the mixture was stirred for 5 minutes and then sonicated without cooling using an ultrasonic rod (Branson Sonifier) for 10 minutes. The solvent was removed on a rotary evaporator. After filtration through a 40 μm filter, the particle size was determined as being 386.9 nm.

Miniemulsion 5

100 g of a polycarbodiimide of the formula I where n=4 and R^{c} is a radical of the formula I′ were mixed with 2.5 g of hexadecane as hydrophobic material and with 400 g of DI water containing 6 g of 15% Steinapol NLS as emulsifier. The mixture was preemulsified at room temperature using an Ultraturrax (IKA T25, 24 000 rpm) for 3 minutes and cleaned by means of a 125 μm pressure filter.

The preemulsion was then passed 3 times with cooling through a high-pressure homogenizer (from Microfluidics). After filtration through a 40 μm filter the particle size was determined as being 368 nm.

IV. Crosslinking Tests

A carboxylate-containing polyurethane dispersion (Luphen® D259U from BASF Aktiengesellschaft) was admixed with increasing amounts of emulsion 1a and then the Fikentscher K value in DMF was measured. Then a film was cast and dried at room temperature for 3 days, after which the measurement was repeated. The K value of the emulsion and of the film is a measure of the crosslinker activity.

The table below shows the results of the K value determinations for different proportions of the individual miniemulsions (emulsions).

TABLE

| Polyurethane | | |
|---|---|---|
| dispersion | K value, dispersion | K value, film |
| 1% s/s emulsion 1a | 45 | undissolved |
| 5% s/s emulsion 1a | 43 | undissolved |
| 10% s/s emulsion 1a | 43 | undissolved |
| 5% s/s emulsion 3 | 49 | undissolved |
| 5% s/s emulsion 5 | 52 | undissolved |

1. An aqueous dispersion comprising
polycarbodiimide having substantially no carboxyl groups and
(i) prepared from polyisocyanate represented by the formula (I)
OCN—(R^{c}—N═C═N)ₙ—R^{c}—NCO (I)
by heating said polyisocyanates, to eliminate carbon dioxide, to form polycarbodiimide
wherein n is from 1 to 20; and
two or more R^{c} radicals present in the same molecule are independently a divalent hydrocarbon radical optionally comprising urea, urethane, ester and/or ether groups obtained by removing the isocyanate groups from a simple organic isocyanate or from a prepolymer which comprises urethane groups, terminal isocyanate groups and optionally ether or ester groups; or
(ii) are represented by formula (II)
R^{d}—(R^{c}—N═C═N—R^{c})ₙ—R^{d} (II)
wherein
n is a number from 1 to 20,
R^{c} is as defined for the formula (I),
R^{d} is —NH₂ 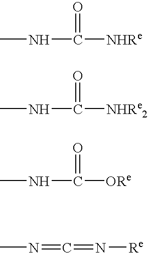
wherein R^{e} is independently C₁ to C₂₀ alkyl or alkenyl, C₅ to C₁₂ cycloalkyl, C₆ to C₂₀ aryl or C₇ to C₂₀ aralkyl, wherein 1 to 4 hydrogen atoms of the radical R^{e} may be substituted by substituents that are inert toward carbodiimide groups and wherein the radical R^{e} does not contain any hydrophilic groups capable of producing dispersibility in water,
and prepared by a process which comprises
heating said polycarbodiimide to form a highly mobile melt, and then emulsifying said melt in water with the aid of emulsifiers.

2. A dispersion as claimed in claim 1, wherein n is from 2 to 10.

3. A dispersion as claimed in claim 1, wherein the radical R^{c} in the formula I is a divalent hydrocarbon radical having from 2 to 50 carbon atoms.

4. A dispersion as claimed in claim 3, wherein the radical R^{c} is represented by formula (I′)

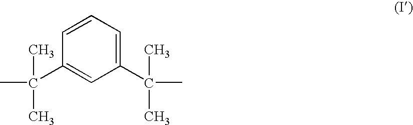

5. A dispersion as claimed in claim 1, wherein the radical R^{c} in the formula (II) is a divalent hydrocarbon radical having from 2 to 50 carbon atoms.

6. A dispersion as claimed in claim 1, wherein the polycarbodiimides of the formula (II) does not have any ionic groups or polyalkylene oxide groups having more than 5 ethylene oxide units.

7. A dispersion as claimed in claim 1, wherein the polycarbodiimides are in the form of a emulsion in water.

8. A dispersion as claimed in claim 7, wherein said emulsion comprises said polycarbodiimides as emulsified particles with a diameter of up to 1 μm in water.

9. A process as claimed in claim 1, wherein said emulsifying comprises treating with ultrasound.

10. A dispersion as claimed in claim 1, wherein the polycarbodiimide prepared from the polyisocyanate is reacted with an alcohol to give a polyurethane having carbodiimide groups.

11. A dispersion as claimed in claim 1, comprising polyurethanes having carbodiimide groups obtained by reacting the polycarbodiimides with alcohols.

12. A binder for coating composition or a binder for impregnating composition which comprises the dispersion as claimed in claim 1.

13. A binder for adhesives, varnishes, paints, papercoating slips or fiber nonwovens which comprises the dispersion as claimed in claim 1.
